SEQUENCE LISTING (3 sequences)

SEQ ID NO: 1 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Adeno-associated virus 2 [sequence omitted]

SEQ ID NO: 3 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

(19) United States
(12) Patent Application Publication
(10) Pub. No.: US 2019/0071681 A1
(43) Pub. Date: Mar. 7, 2019

(54) AAV HEPARIN MUTANTS THAT DISPLAY SIGNIFICANTLY IMPROVED EYE AND BRAIN TRANSDUCTION

(71) Applicant: University of Florida Research Foundation, Incorporated, Gainesville, FL (US)

(72) Inventors: Nicholas Muzyczka, Gainesvile, FL (US); Kenneth H. Warrington, Buffalo, NY (US); Marina Gorbatyuk, Birmingham, AL (US); Oleg Gorbatyuk, Birmingham, AL (US)

(73) Assignee: University of Florida Research Foundation, Incorporated, Gainesville, FL (US)

(21) Appl. No.: 16/079,825

(22) PCT Filed: Feb. 24, 2017
(86) PCT No.: PCT/US17/19436

§ 371 Date: Aug. 24, 2018

Related U.S. Application Data
(60) Provisional application No. 62/300,739, filed on Feb. 26, 2016.

(51) Int. Cl. C12N 15/66; C07K 14/005; C12N 15/86; A61K 9/00

(52) U.S. Cl. CPC C12N 15/66; C07K 14/005; C12N 15/86; C12N 2750/14145; A61K 9/0085; C12N 2750/14122; C12N 2750/14143; A61K 9/0048

(57) ABSTRACT

Disclosed are methods of gene delivery using capsid-modified recombinant adeno-associated viral (rAAV) particles. Exemplary methods are provided employing rAAV particles that have altered affinity for heparin or heparin sulfate. Also provided by the disclosure are methods employing the rAAV vector-based compositions, virus particles, host cells, and pharmaceutical formulations in the expression of selected therapeutic genes, proteins, polypeptides, peptides, antisense oligonucleotides, and/or ribozymes in selected mammals, including organs, tissues, and human host cells.

### CROSS-REFERENCE TO RELATED APPLICATIONS

This application claims the benefit of U.S. Provisional Patent Application No. 62/300,739, filed Feb. 26, 2016, the contents of which are incorporated herein by reference in their entirety.

### GOVERNMENT SUPPORT

This invention was made with government support under HL059412, NS063602, and NS069574 awarded by the National Institutes of Health. The government has certain rights in the invention.

### FIELD

The present disclosure relates generally to the fields of molecular biology and virology, and in particular, to the development of gene delivery vehicles. The disclosure provides improved recombinant adeno-associated virus (rAAV) vectors that comprise modifications that result in enhanced transduction efficiencies when compared to unmodified vectors.

### SUMMARY

Aspects of the application relate to a method of delivering a gene of interest to a cell of the brain or eye, the method comprising providing to the cell a composition comprising a recombinant adeno-associated virus (rAAV) particle comprising an AAV capsid protein having an amino acid substitution at one or more positions selected from R484, R487, K532, R585, and R588 (e.g., one or more of the following amino acid substitutions R484A, R487A, K532A, R585A, and/or R588A). In some embodiments, the rAAV particle is derived from an AAV2 serotype. In some embodiments, the rAAV particle is derived from an AAV1 or AAV3 serotype. In some embodiments, the rAAV particle comprises a nucleic acid encoding the gene of interest.

In some embodiments, the gene of interest is a therapeutic gene. In some embodiments, the therapeutic gene encodes a therapeutic polypeptide or a therapeutic protein. In some embodiments, the therapeutic gene encodes a therapeutic ribonucleic acid (RNA). In some embodiments, the RNA comprises mRNA, tRNA, rRNA, siRNA, microRNA, antisense RNA, or a ribozyme. In some embodiments, the therapeutic gene is a brain-specific gene or an eye-specific gene.

In some embodiments, the therapeutic gene comprises brain-derived neurotrophic factor (BDNF), tyrosine hydroxylase, aromatic amino acid decarboxylase, β-glucuronidase, exosaminidase A, herpes simplex virus, or thymidine kinase. In some embodiments, the therapeutic gene comprises opsin protein of rhodopsin (RHO), cyclic GMP phosophodiesterase α-subunit (PDE6A) or β-subunit (PDE6B), alpha subunit of the rod cyclic nucleotide gated channel (CNGA1), RPE65, RLBP1, ABCR, peripherin/RDS, ROM1, arrestin (SAG), alpha-transducin (GNAT1), rhodopsin kinase (RHOK), guanylate cyclase activator 1A (GUCA1A), retina specific guanylate cyclase (GUCY2D), alpha subunit of the cone cyclic nucleotide gated cation channel (CNGA3), BCP cone opsin gene, GCP cone opsin gene, or RCP cone opsin gene.

In some aspects, the disclosure relates to an rAAV particle comprising: a) an AAV capsid protein having an amino acid substitution at one or more positions selected from R484, R487, K532, R585, and R588 (e.g., one or more of the following amino acid substitutions R484A, R487A, K532A, R585A, and/or R588A); and b) a brain-specific or eye-specific gene of interest, or a gene of interest operatively connected to a brain-specific or eye-specific promoter. In some aspects, the disclosure relates to a composition comprising the rAAV particle. In some embodiments, the gene of interest is flanked by AAV inverted terminal repeats (ITRs).

Further aspects of the disclosure relate to a method of targeting the brain or eye of a subject, the method comprising administering to the subject a composition comprising a recombinant adeno-associated virus (rAAV) particle comprising an AAV capsid protein having an amino acid substitution at one or more positions selected from R484, R487, K532, R585, and R588 (e.g., one or more of the following amino acid substitutions R484A, R487A, K532A, R585A, and/or R588A). In some embodiments, the composition is administered (e.g., by injection) subcutaneously, intraocularly, intravitreally, parenterally, subcutaneously, intravenously, intracranially, intracerebrally, intracerebro-ventricularly, intramuscularly, intrathecally, orally, intraperitoneally, intraspinally, epidurally, intradurally, subdurally, retrobulbarly, ophthalmicly, subretinally, intracorneally, conjunctivally, directly to the brain, directly to the CNS, directly to the peripheral nervous system, subconjunctivally, by oral or nasal inhalation, or by direct injection to one or more cells, tissues, or organs. In some embodiments, the subject has a brain or eye condition, disease, or disorder.

### BRIEF DESCRIPTION OF THE DRAWINGS

The following drawings form part of the present specification and are included to further demonstrate certain aspects of the present disclosure. The disclosure may be better understood by reference to the following description taken in conjunction with the accompanying drawings, in which like reference numerals identify like elements, and in which:

FIG. 1. Viral genome copy numbers in selected tissues after tail vein (TV) injection from an exemplary set of results are depicted. GFP genome copy numbers perm of DNA in selected tissues at 4 weeks after tail vein injection of wild type (wt) and capsid mutant viral vectors. One-way ANOVA analysis. Tukey post hoc results are indicated as *, ** and ***=P<0.05, 0.01 and 0.001, respectively vs. wt AAV2; N=4 per group.

FIG. 2. Exemplary confocal images of the striatum after injection of wild type rAAV2 (wt) and its capsid mutant viral vectors are depicted. NeuN and GFP merged fluorescent images demonstrate distribution area transduced with each viral vector in the striatum (FIG. 2A, E, I, M). Sections were visualized for native GFP and immunostained for NeuN and GFAP. Images shown in (FIG. 2B, F, J, N) demonstrate GFP alone. Corresponding merge images show GFP and NeuN (FIG. 2C, G, K, and O) as well as GFP and GFAP (FIGS. 2D, H, L and P). Scale bars: (A, E, I, M) 1 mm, (B-D, F-H, J-L, N-P) 50 μm.

FIG. 3. Exemplary confocal images illustrate intensity level of GFP fluorescence produced by wt and capsid mutant viral vectors in the striatum. Striatal section taken at the level of the site of injection were analyzed under confocal microscope with gradual level of laser bulb power (100%, 30%, 10%, 3% and 1%) at the same setting for all viral vectors.

FIG. 4. A non-limiting quantitative analysis of GFP-positive cells in the brain at 4 weeks after rAAV injections is depicted. Montages of rostral-to-caudal coronal sections illustrate the extent of expression of GFP in the brain after bi-lateral injections with wt and capsid mutant viral vectors (FIG. 4A). Outlined areas (in μm²) containing GFP-positive cells on every 8 serial section were delineated for unbiased stereology count and demonstrate comparative distribution extent of cell infected with wt and capsid mutant viruses (FIG. 4B). Number of cells transduced with each virus estimated from stereological counts (FIG. 4C). Volume of distribution through the brain tissue transduced with wt and capsid mutant viruses (FIG. 4D). Number of cells transduced per volume (mm³) calculated from above measurements is shown in FIG. 4E.

FIG. 5. Exemplary antero-retrograde transport of wt rAAV and mutants is depicted, with merged views showing TH-positive neurons that have been transduced with GFP. GFP tracing of the anterograde projections to the substantia nigra pars reticulate (SNr) (FIG. 5A). Retrograde transport of R585A mutant in the SNc (FIG. 5B). Confocal image demonstrates a selective transduction of dopaminergic neurons in the SNc after R585, 588A mutant injection into the striatum (FIG. 5C). Only single GFP expressing nigral neurons were detected with standard immune-peroxidase method in rats injected with R484, 585, 588A mutant virus (FIG. 5D). Scale bars: 50 μm.

FIG. 6. Exemplary confocal images of retinas injected with wt and capsid mutant rAAVs are shown. Full retina mapping was conducted using propidium iodide (PI) nuclear staining and GFP native fluorescence (FIGS. 6A, D, G, and J). GFP fluorescence observed in expressed cells of representative sectors (FIGS. 6B, E, H and K). Propidium iodide and GFP fluorescence merged images of representative sectors (FIGS. 6C, F, I and L).

FIG. 7. Exemplary unbiased stereology count of photoreceptor cell nuclei. Two-fold difference was found in mouse retinas injected with AAV2 R585, 588A compared with wt rAAV and mutants.

### DETAILED DESCRIPTION

Aspects of the application relate to the use of one or more heparin sulfate proteoglycan (HSPG) binding-deficient AAV variants to target cells or tissue of the brain and eyes. Accordingly, such AAV variants can be used to deliver one or more genes of interest to brain and/or eye tissue (e.g., in a human subject). Aspects of the application are based, at least in part, on the surprising discovery that HSPG binding-deficient AAV variants are concentrated in the brain and eye after administration to a subject.

HSPG binding-deficient AAV variants that can be used include an AAV capsid protein having an amino acid substitution at one or more of positions R484, R487, K532, R585, and R588 (e.g., in an AAV2 capsid protein), or at one or more positions corresponding to positions R484, R487, K532, R585, and R588 in an AAV2 capsid protein (e.g., in a capsid protein of a different serotype). In some embodiments, the amino acid substitution comprises alanine substitution. In some embodiments, the variants include one or more of the amino acid substitutions comprising R484A, R487A, K532A, R585A, and R588A. In some embodiments, the variant comprises R484A, R487A, K532A, R585A, and R588A. In some embodiments, the variant comprises R484A. In some embodiments, the variant comprises R487A. In some embodiments, the variant comprises K532A. In some embodiments, the variant comprises R585A. In some embodiments, the variant comprises R588A. In some embodiments, the variant comprises R484A and R487A. In some embodiments, the variant comprises R484A and K532A. In some embodiments, the variant comprises R484A and R585A. In some embodiments, the variant comprises R484A and R588A. In some embodiments, the variant comprises R487A and K532A. In some embodiments, the variant comprises R487A and R585A. In some embodiments, the variant comprises R487A and R588A. In some embodiments, the variant comprises K532A and R585A. In some embodiments, the variant comprises K532A and R588A. In some embodiments, the variant comprises R585A and R588A. In some embodiments, the variant comprises R484A, R487A, and K532A. In some embodiments, the variant comprises R484A, R487A, and R585A. In some embodiments, the variant comprises R484A, R487A, and R588A. In some embodiments, the variant comprises R484A, K532A, and R585A. In some embodiments, the variant comprises R484A, K532A, and R588A. In some embodiments, the variant comprises R484A, R585A, and R588A. In some embodiments, the variant comprises R487A, K532A, and R585A. In some embodiments, the variant comprises R487A, K532A, and R588A. In some embodiments, the variant comprises R487A, R585A, and R588A. In some embodiments, the variant comprises K532A, R585A, and R588A. In some embodiments, the variant comprises R484A, R487A, K532A, and R585A. In some embodiments, the variant comprises R484A, R487A, K532A, and R588A. In some embodiments, the variant comprises R484A, R487A, R585A, and R588A. In some embodiments, the variant comprises R484A, K532A, R585A, and R588A. In some embodiments, the variant comprises R487A, K532A, R585A, and R588A. In some embodiments, one or more arginine or lysine amino acids independently can be substituted by an amino acid other than Alanine. In some embodiments, the amino acid other than Alanine is a conservative substitution of Alanine (for example Glycine). However, in some embodiments one or more arginine or lysine amino acids independently can be substituted by an amino acid from any of the following groups of amino acids: (a) methionine, isoleucine, leucine, valine; (b) phenylalinine, tyrosine, tryptophan; (c) serine, threonine; (d) glutamine, asparagine; and (e) glutamic acid, aspartic acid. In some embodiments, the amino acids may be modified. In some embodiments, the amino acid substitution includes analogs that include residues other than naturally occurring L-amino acids, e.g., D-amino acids or non-naturally occurring or synthetic amino acids, e.g., β or γ amino acids; and cyclic analogs. In some embodiments, the amino acid substitutions do not promote or support heparin binding. For example, an rAAV particle comprising one or more of these subsitutions may have less than 90%, less than 80%, less than 70%, less than 60%, less than 50%, less than 40%, less than 30%, less than 20% or less than 10% of the heparin binding activity of a corresponding wildtype particle.

In some aspects, HSPG binding-deficient AAV variants can be used to deliver one or more genes of interest under the control of one or more promoters of interest. In some embodiments, genes of interest can be genes that provide a missing or therapeutic function in brain or eye tissue (e.g., brain- or eye-specific genes). In some embodiments, genes encoding functional elements of the eye or brain are useful. Non-limiting exemplary genes of interest include brain-derived neurotrophic factor (BDNF) for treatment of neurodegenerative disease, stroke, or brain trauma; tyrosine hydroxylase and/or aromatic amino acid decarboxylase for Parkinson's disease; β-glucuronidase; hexosaminidase A; herpes simplex virus thymidine kinase or genes encoding antisense RNA to the epidemal growth factor receptor for treatment of brain tumors; lysosomal storage disorder replacement enzymes for Tay-Sachs and other lysosomal storage disorders; gene encoding antisense RNA for the treatment of the cerebral component of acquired immune deficiency syndrome (AIDS). Eye-specific therapeutic genes include opsin protein of rhodopsin (RHO), cyclic GMP phosophodiesterase α-subunit (PDE6A) or β-subunit (PDE6B), the alpha subunit of the rod cyclic nucleotide gated channel (CNGA1), RPE65, RLBP1, ABCR, peripherin/RDS, ROM1, and arrestin (SAG), which are all known to be mutated in RP. In addition, other genes are mutated in RP-related disorders, including alpha-transducin (GNAT1), rhodopsin kinase (RHOK), guanylate cyclase activator 1A (GUCA1A), retina specific guanylate cyclase (GUCY2D), the alpha subunit of the cone cyclic nucleotide gated cation channel (CNGA3), and cone opsin genes such as BCP, GCP, and RCP, which are mutated in certain forms of color blindness. In some embodiments, one or more genes of interest are under the control or one or more promoters of interest.

Accordingly, promoters of interest include promoters that are either active or selectively active in brain or eye tissue (e.g., brain- or eye-specific promoters). Non-limiting exemplary promoters of interest include, but are not limited to, Immunoglobulin Heavy Chain, Immunoglobulin Light Chain, T Cell Receptor, HLA DQ a and DQ β, β-Interferon, Interleukin-2, Interleukin-2 Receptor, MHC Class II 5, MHC Class II HLA-Dra, β-Actin, Muscle Creatine Kinase, Prealbumin (Transthyretin), Elastase I, Metallothionein, Collagenase, Albumin Gene, α-Fetoprotein, t-Globin, β-Globin, e-fos, c-HA-ras, Insulin, Neural Cell Adhesion Molecule (NCAM), al-Antitrypain, H2B (TH2B) Histone, Mouse or Type I Collagen, Glucose-Regulated Proteins (GRP94 and GRP78), Rat Growth Hormone, Human Serum Amyloid A (SAA), Troponin I (TN I), Platelet-Derived Growth Factor, Duchenne Muscular Dystrophy, SV40, Polyoma, Retroviruses, Papilloma Virus, Hepatitis B Virus, Human Immunodeficiency Virus, Cytomegalovirus, Gibbon Ape Leukemia Virus.

Accordingly, aspects of the AAV variants described herein can be useful for delivering one or more genes of interest (e.g., one or more genes of interest under the control of one or more promoters of interest) to one or more cell types of the brain and/or eye. Exemplary cell types include cells located in the ganglion cell layer (GCL), the inner plexiform layer inner (IPL), the inner nuclear layer (INL), the outer plexiform layer (OPL), outer nuclear layer (ONL), outer segments (OS) of rods and cones, the retinal pigmented epithelium (RPE), the inner segments (IS) of rods and cones, the epithelium of the conjunctiva, the iris, the ciliary body, the corneum, epithelium of ocular sebaceous glands, neurons, glial cells, pericytes, smooth muscle cells, microglia, Schwann cells, oligodendrocytes, and astrocytes.

Aspects of the disclosure provide HSPG binding-deficient AAV variants that are useful for targeting one or more cell types of the brain and/or eye. Accordingly, the AAV variants of the disclosure can be useful in treating one or more conditions of interest (e.g., diseases and disorders). Exemplary conditions of interest which are amenable to treatment according to the methods of the disclosure include, but are not necessarily limited to, retinitis pigmentosa (RP), diabetic retinopathy, and glaucoma, including open-angle glaucoma (e.g., primary open-angle glaucoma), angle-closure glaucoma, and secondary glaucomas (e.g., pigmentary glaucoma, pseudoexfoliative glaucoma, and glaucomas resulting from trauma and inflammatory diseases). Further exemplary conditions amenable to treatment according to the disclosure include, but are not necessarily limited to, retinal detachment, age-related or other maculopathies, photic retinopathies, surgery-induced retinopathies, toxic retinopathies, retinopathy of prematurity, retinopathies due to trauma or penetrating lesions of the eye, inherited retinal degenerations, surgery-induced retinopathies, toxic retinopathies, retinopathies due to trauma or penetrating lesions of the eye.

Exemplary inherited conditions of interest for treatment according to the disclosure include, but are not necessarily limited to, Bardet-Biedl syndrome (autosomal recessive); Congenital amaurosis (autosomal recessive); Cone or cone-rod dystrophy (autosomal dominant and X-linked forms); Congenital stationary night blindness (autosomal dominant, autosomal recessive and X-linked forms); Macular degeneration (autosomal dominant and autosomal recessive forms); Optic atrophy, autosomal dominant and X-linked forms); Retinitis pigmentosa (autosomal dominant, autosomal recessive and X-linked forms); Syndromic or systemic retinopathy (autosomal dominant, autosomal recessive and X-linked forms); and Usher syndrome (autosomal recessive).

Further exemplary conditions of interest which are amenable to treatment according to the methods of the disclosure include, but are not necessarily limited to, spinal cord injury and/or motor neuron diseases, Parkinson's disease, epilepsy, and seizures. In some embodiments, conditions of interest include neurological conditions. In some embodiments, conditions of interest include neurodegenerative conditions. In some embodiments, conditions of interest include alcoholism, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), chronic pain, Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, and Tables dorsalis.

Further exemplary conditions of interest which are amenable to treatment according to the methods of the disclosure include, but are not necessarily limited to, neurodevelopmental disorders. In some embodiments, conditions of interest include attention deficit hyperactivity disorder (ADHD), attention deficit disorder (ADD), schizophrenia, obsessive-compulsive disorder (OCD), mental retardation, autistic spectrum disorders (ASD), cerebral palsy, Fragile-X Syndrome, Downs Syndrome, Rett's Syndrome, Asperger's syndrome, Williams-Beuren Syndrome, childhood disintegrative disorder, articulation disorder, learning disabilities, dyslexia, expressive language disorder, and mixed receptive-expressive language disorder, verbal or performance aptitude. In some embodiments, conditions of interest include bi-polar disorders, anorexia, general depression, seizures, obsessive compulsive disorder (OCD), anxiety, bruixism, Angleman's syndrome, aggression, explosive outburst, self injury, post traumatic stress, conduct disorders, Tourette's disorder, stereotypic movement disorder, mood disorder, sleep apnea, restless legs syndrome, dysomnias, paranoid personality disorder, schizoid personality disorder, schizotypal personality disorder, antisocial personality disorder, borderline personality disorder, histrionic personality disorder, narcissistic personality disorder, avoidant personality disorder, dependent personality disorder, reactive attachment disorder; separation anxiety disorder; oppositional defiant disorder; dyspareunia, pyromania, kleptomania, trichotillomania, gambling, pica, neurotic disorders, alcohol-related disorders, amphetamine-related disorders, cocaine-related disorders, marijuana abuse, opioid-related disorders, phencyclidine abuse, tobacco use disorder, bulimia nervosa, delusional disorder, sexual disorders, phobias, somatization disorder, enuresis, encopresis, disorder of written expression, expressive language disorder, mental retardation, mathematics disorder, transient tic disorder, stuttering, selective mutism, Crohn's disease, ulcerative colitis, bacterial overgrowth syndrome, carbohydrate intolerance, celiac sprue, infection and infestation, intestinal lymphangiectasia, short bowel syndrome, tropical sprue, Whipple's disease, Alzheimer's disease, Parkinson's Disease, ALS, spinal muscular atrophies, and Huntington's Disease.

Accordingly, compositions herein can be administered to a subject in need of treatment. In some embodiments, the subject has or is suspected of having one or more conditions, diseases, or disorders of the brain and/or eye. In some embodiments, the subject has or is suspected of having one or more of the conditions, diseases, and disorders disclosed herein. In some embodiments, the subject is a human. In some embodiments, the subject is a non-human primate. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. Other exemplary subjects include domesticated animals such as dogs and cats; livestock such as horses, cattle, pigs, sheep, goats, and chickens; and other animals such as mice, rats, guinea pigs, and hamsters.

In some embodiments, the dose of rAAV particles administered to a cell or a subject may be on the order ranging from 10⁶ to 10¹⁴ particles/mL or 10³ to 10¹⁵ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the dose of rAAV particles administered to a subject may be on the order ranging from 10⁶ to 10¹⁴ vector genomes (vgs)/mL or 10³ to 10¹⁵ vgs/mL, or any values therebetween for either range, such as for example, about 10⁶, 10⁷, 10⁸, 10⁹, 10¹⁰, 10¹¹, 10¹², 10¹³, or 10¹⁴ vgs/mL. In one embodiment, rAAV particles of higher than 10¹³ vgs/mL are be administered. The rAAV particles can be administered as a single dose, or divided into two or more administrations as may be required to achieve therapy of the particular disease or disorder being treated. In some embodiments, 0.0001 mL to 10 mLs are delivered to a subject.

In some embodiments, the disclosure provides formulations of one or more rAAV-based compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or nucleic acid vectors may be administered in combination with other agents as well, such as, e.g., particles, proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or host cell) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver an rAAV particle or host cell in suitably formulated pharmaceutical compositions disclosed herein either subcutaneously, intraocularly, intravitreally, parenterally, subcutaneously, intravenously, intracranially, intracerebrally, intracerebro-ventricularly, intramuscularly, intrathecally, orally, intraperitoneally, intraspinally, epidurally, intradurally, subdurally, retrobulbarly, ophthalmicly, subretinally, intracorneally, conjunctivally, directly to the brain, directly to the CNS, directly to the peripheral nervous system, subconjunctivally, by oral or nasal inhalation, or by direct injection to one or more cells, tissues, or organs.

The pharmaceutical forms of the rAAV particle or host cell compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The term “carrier” refers to a diluent, adjuvant, excipient, or vehicle with which the rAAV particle or host cell is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

The compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

The compositions of the present disclosure can be delivered to the eye through a variety of routes. They may be delivered intraocularly, by topical application to the eye or by intraocular injection into, for example the vitreous (intravitreal injection) or subretinal (subretinal injection) inter-photoreceptor space. Alternatively, they may be delivered locally by insertion or injection into the tissue surrounding the eye. They may be delivered systemically through an oral route or by subcutaneous, intravenous or intramuscular injection. Alternatively, they may be delivered by means of a catheter or by means of an implant, wherein such an implant is made of a porous, non-porous or gelatinous material, including membranes such as silastic membranes or fibers, biodegradable polymers, or proteinaceous material. They can be administered prior to the onset of the condition, to prevent its occurrence, for example, during surgery on the eye, or immediately after the onset of the pathological condition or during the occurrence of an acute or protracted condition.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or host cells in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of rAAV particle, nucleic acid vector, or host cell compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or host cell compositions, either over a relatively short, or a relatively prolonged period of time, as may be determined by the medical practitioner overseeing the administration of such compositions.

The composition may include rAAV particles or host cells, either alone, or in combination with one or more additional active ingredients, which may be obtained from natural or recombinant sources or chemically synthesized. In some embodiments, rAAV particles are administered in combination, either in the same composition or administered as part of the same treatment regimen, with a proteasome inhibitor, such as Bortezomib, or hydroxyurea.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Aspects of the disclosure relate to recombinant adeno-associated virus (rAAV) particles for delivery of one or more nucleic acid vectors comprising a gene of interest into various tissues, organs, and/or cells. In some embodiments, the rAAV particles comprise an rAAV capsid protein as described herein, e.g., comprising one or more amino acid substitutions. In some embodiments, the gene of interest encodes a polypeptide or protein of interest (e.g., a therapeutic polypeptide or protein). In some embodiments, the gene of interest encodes an RNA of interest (e.g., a therapeutic mRNA, siRNA, microRNA, antisense RNA, tRNA, rRNA, or a ribozyme). In some embodiments, a gene of interest is a brain-specific gene. In some embodiments, a gene of interest is an eye-specific gene.

Recombinant AAV (rAAV) particles may comprise at a minimum (a) one or more heterologous nucleic acid regions comprising a sequence encoding a gene of interest (e.g., a protein of interest or an RNA of interest) and (b) one or more regions comprising inverted terminal repeat (ITR) sequences (e.g., wild-type ITR sequences or engineered ITR sequences) flanking the one or more heterologous nucleic acid regions. In some embodiments, the nucleic acid vector is between 4 kb and 5 kb in size (e.g., 4.2 to 4.7 kb in size). This nucleic acid vector may be encapsidated by a viral capsid, such as an AAV1, AAV2, or AAV3 capsid, which may comprise a modified capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complementary vector that contains a region of the nucleic acid vector that is complementary to another region of the nucleic acid vector, initiating the formation of the double-strandedness of the nucleic acid vector.

The rAAV particle may be of any AAV serotype, including any derivative or pseudotype (e.g., 1, 2, 3, 4, 5, 6, 7, 8, 9, 10, 2/1, 2/5, 2/8, or 2/9). As used herein, the serotype of an rAAV viral vector (e.g., an rAAV particle) refers to the serotype of the capsid proteins of the recombinant virus. In some embodiments, the rAAV particle is not AAV2. In some embodiments, the rAAV particle is AAV2. In some embodiments, the rAAV particle is AAV6. In some embodiments, the rAAV particle is an AAV6 serotype comprising an rAAV capsid protein as described herein. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). In some embodiments, the rAAV particle is a pseudotyped rAAV particle, which comprises (a) a nucleic acid vector comprising ITRs from one serotype (e.g., AAV2) and (b) a capsid comprised of capsid proteins derived from another serotype (e.g., AAV1, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

In some embodiments, the rAAV particle comprises a capsid that includes modified capsid proteins (e.g., capsid proteins comprising a modified VP3 region) optionally further modified to replace one or more surface exposed lysine or arginine residues (e.g., in a VP3 region of a capsid protein, see, e.g., U.S Patent Publication Number US20130310443, which is incorporated herein by reference in its entirety). In some embodiments, the rAAV particle comprises a modified capsid protein comprising a non-arginine residue (e.g., an alanine) at a position that corresponds to a surface-exposed arginine residue in a wild-type capsid protein, a non-lysine residue (e.g., an alanine) at a position that corresponds to a surface-exposed lysine residue in the wild-type capsid protein, or a combination thereof. Exemplary surface-exposed residues include positions that correspond to R484, R487, K532, R585, or R588 of the wild-type AAV2 capsid protein. In some embodiments, the AAV variant comprises an AAV1 or AAV3 capsid protein. It should be appreciated that in such embodiments, the AAV variant may comprise one or more amino acid substitutions at positions corresponding to R484, R487, K532, R585, and R588 of AAV2. Exemplary, non-limiting wild-type capsid protein sequences are provided below (SEQ ID NOs: 1-3).

| Exemplary AAV1 capsid protein | |
|---|---|
| (SEQ ID NO: 1) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEEVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD EDKFFPMSGV MIFGKESAGA |
| 551 | SNTALDNVMI TDEEEIKATN PVATERFGTV AVNFQSSSTD PATGDVHAMG |
| 601 | ALPGMVWQDR DVYLQGPIWA KIPHTDGHFH PSPLMGGFGL KNPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL* |
| Exemplary AAV2 capsid protein | |
| (SEQ ID NO: 2) | |
| 1 | MAADGYLPDW LEDTLSEGIR QWWKLKPGPP PPKPAERHKD DSRGLVLPGY |
| 51 | KYLGPFNGLD KGEPVNEADA AALEHDKAYD RQLDSGDNPY LKYNHADAEF |
| 101 | QERLKEDTSF GGNLGRAVFQ AKKRVLEPLG LVEEPVKTAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TTSTRTWALP |
| 251 | TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRTNT |
| 451 | PSGTTTQSRL QFSQAGASDI RDQSRNWLPG PCYRQQRVSK TSADNNNSEY |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDTNGVY SEPRPIGTRY LTRNL* |
| Exemplary AAV3 capsid protein | |
| (SEQ ID NO: 3) | |
| 1 | MAADGYLPDW LEDNLSEGIR EWWALKPGVP QPKANQQHQD NRRGLVLPGY |
| 51 | KYLGPGNGLD KGEPVNEADA AALEHDKAYD QQLKAGDNPY LKYNHADAEF |
| 101 | QERLQEDTSF GGNLGRAVFQ AKKRILEPLG LVEEAAKTAP GKKGAVDQSP |
| 151 | QEPDSSSGVG KSGKQPARKR LNFGQTGDSE SVPDPQPLGE PPAAPTSLGS |
| 201 | NTMASGGGAP MADNNEGADG VGNSSGNWHC DSQWLGDRVI TTSTRTWALP |
| 251 | TYNNHLYKQI SSQSGASNDN HYFGYSTPWG YFDFNRFHCH FSPRDWQRLI |
| 301 | NNNWGFRPKK LSFKLFNIQV RGVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFQ FSYTFEDVPF HSSYAHSQSL DRLMNPLIDQ YLYYLNRTQG |
| 451 | TTSGTTNQSR LLFSQAGPQS MSLQARNWLP GPCYRQQRLS KTANDNNNSN |
| 501 | FPWTAASKYH LNGRDSLVNP GPAMASHKDD EEKFFPMHGN LIFGKEGTTA |
| 551 | SNAELDNVMI TDEEEIRTTN PVATEQYGTV ANNLQSSNTA PTTGTVNHQG |
| 601 | ALPGMVWQDR DVYLQGPIWA KIPHTDGHFH PSPLMGGFGL KHPPPQIMIK |
| 651 | NTPVPANPPT TFSPAKFASF ITQYSTGQVS VEIEWELQKE NSKRWNPEIQ |
| 701 | YTSNYNKSVN VDFTVDTNGV YSEPRPIGTR YLTRNL* |

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid containing the nucleic acid vector may be combined with one or more helper plasmids, e.g., that contain a rep gene (e.g., encoding Rep78, Rep68, Rep52 and Rep40) and a cap gene (e.g., encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene (e.g., encoding a rAAV capsid protein as described herein) and a second helper plasmid comprising a Ela gene, a E1b gene, a E4 gene, a E2a gene, and a VA gene. In some embodiments, the rep gene is a rep gene derived from AAV2 or AAV6 and the cap gene is derived from AAV2 or AAV6 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

The disclosure also contemplates host cells that comprise at least one of the disclosed rAAV particles or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself. In some embodiments, the host cell is a cell of erythroid lineage, such as a CD36⁺ burst-forming units-erythroid (BFU-E) cell or a colony-forming unit-erythroid (CFUE-E) progenitor cell.

Illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Biodistribution of Wild-Type and Variant rAAV

AAV2 utilizes the heparan sulfate proteoglycan (HSPG) as its primary receptor, which is abundantly expressed by most neuronal cells and other cell types. HSPG is a component of the extracellular matrix. Taking this into account, it was successfully demonstrated that co-administration of AAV2 with heparin or mannitol can significantly increase the transduction region. Another possible strategy is based on point mutations in receptor binding residues of viral capsids. Five amino acids have been identified, namely, arginines 484, 487, 585, and 588 and one lysine at position 532, that mediate the natural affinity of AAV2 for heparan sulfate glucosaminoglycans (HSGAG).

Three AAV2 capsid mutants were generated, in which charged-to-alanine substitutions were made in VP3 at the following positions: R585A (single mutation), R585A and R588A (double mutant), and R484A, R585A and R588A (triple mutant). All viral vectors contained a CMVenhancer/chicken β-actin promoter driving the expression of green fluorescent protein (GFP) gene.

To validate comparative virus distribution and expression level, wild type (wt) and mutant capsid rAAV2s were injected into mouse tail vein and the retina as well as into the rat striatum. The efficacy of each was analyzed at 4 weeks after virus administration, which is based on observations that protein expression of rAAV mediated genes reach a maximum at 3-4 weeks after virus application.

Based on a previous study evaluating the role of particular amino acids in HSGAG receptor binding, three rAAV2 capsid mutants were generated to evaluate tropism and efficiency of delivered gene expression. Among a panel of analyzed mutants, charged-to-alanine substitutions were made in positions R585A (single mutant), R585A and R588A (double mutant), and R484A, R585A and R588A (triple mutant). The packaging vector contained the coding sequence for humanized GFP driven by synthetic CBA promoter with a cytomegalovirus (CMV) immediate-early enhancer. Three routes of viral application were used in this study: tail vein, subretinal, and brain (striatum) injections. All animals were sacrificed at 4 weeks after virus administration. Four weeks time point was selected based on previous studies demonstrating that this point correlates with maximum expression level of rAAV mediated vectors.

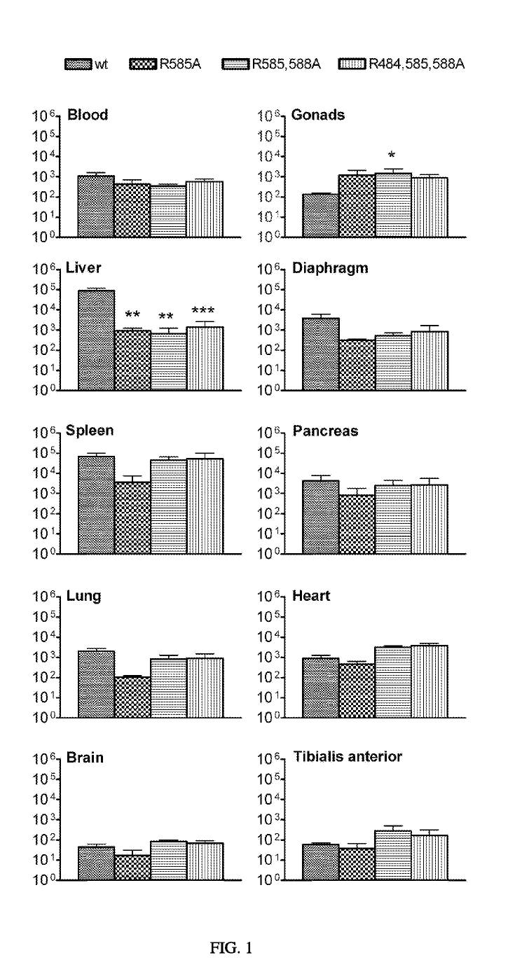

The viral genome copy number remaining in the blood of mice injected with wt rAAV2 and capsid mutants was assessed. The results revealed that each of the examined viral vectors was showing a similar amount of viral genomes remaining in the blood. FIG. 1 depicts the viral genome copy numbers remaining in selected tissue samples using real-time-PCR. These results did not reveal any significant differences in expression level between injected groups in most of the tissue samples examined. The liver was the only tissue in which a significant difference in expression levels was observed, with wt rAAV2 being significantly higher than the mutants used in this study (FIG. 1). Viral genome copy number for injected viral groups did not exceed 10⁴/m in most tissues examined, except of wt, R585, 588A, and R484, 585, 588A in the spleen (>10⁴) as well as wt rAAV2 in the liver (>10⁵). Very low level of expression was identified in the brain and muscles (tibialis anterior), which did not exceeded 10² copies (FIG. 1).

### Example 2: Wild-Type and Variant rAAV Variants Localize to Neuronal Cells of Rat Striata

The striatum (ST) is a large structure in the rat brain, and previous work found that more than one injection of rAAV2/2 is necessary to transduce most of the region and obtain a change in phenotype relevant to Parkinson disease. Therefore, it was desirable to investigate the extent of transduction of rAAV2/1 and rAAV2/5 compared to rAAV2/2 in this brain region. It was found that both rAAV2/1 and rAAV2/5 transduced a significantly larger number of cells.

The ST was selected to investigate the extent of transduction of wt rAAV2 and capsid viral mutants. It was previously found that administration of wt rAAV results in neuronal transduction of restricted areas in the ST. Therefore, it was desirable to determine if structural changes in HSGAG receptor binding motif are able to significantly modulate the extent of neuronal transduction.

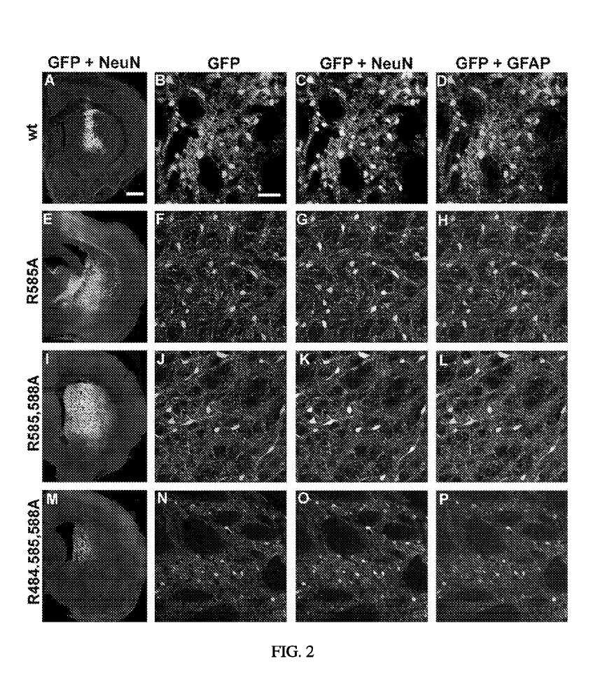

Confocal microscopy analysis of brain sections obtained from rats injected with viral vectors into the striatum discovered visually discernible differences in a number of infected cells, transduction volume, as well as in the level of GFP expression between wt rAAV and capsid mutants (FIG. 2A, E, I, M). Immunocytochemistry with an antibody specific to the neuronal marker NeuN and antibodies against the astrocyte marker GFAP revealed that most, if not all, GFP expressing cells were also positive for NeuN (FIGS. 2B and C, F and G, J and K, N and O). In contrast, no overlap between GFP-positive cells and GFAP staining was identified after injection of either wt rAAV2 or the capsid mutants (FIGS. 2B and D, F and H, J and L, N and P).

### Example 3: rAAV Variants Transduce Tissue More Effectively than the Wild-Type

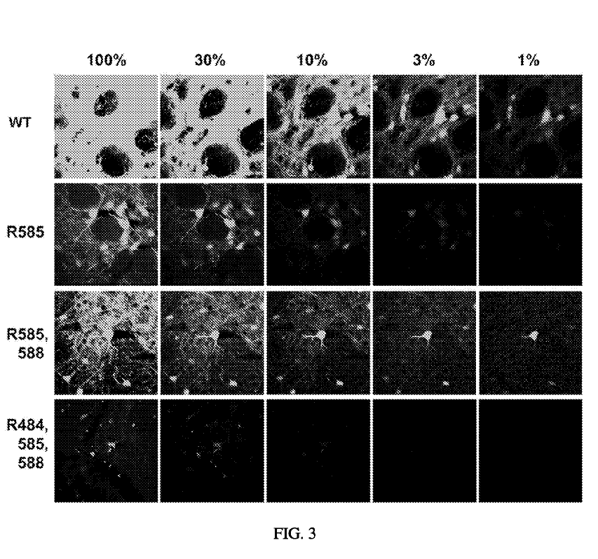

It was found that all three mutants transduced a significantly greater tissue volume and larger number of neurons than wt AAV2 (FIG. 3) Among the mutants, stereology count revealed a higher number of GFP-positive cells in R585,R588A injected animals when compared to either the single mutant or the triple mutant.

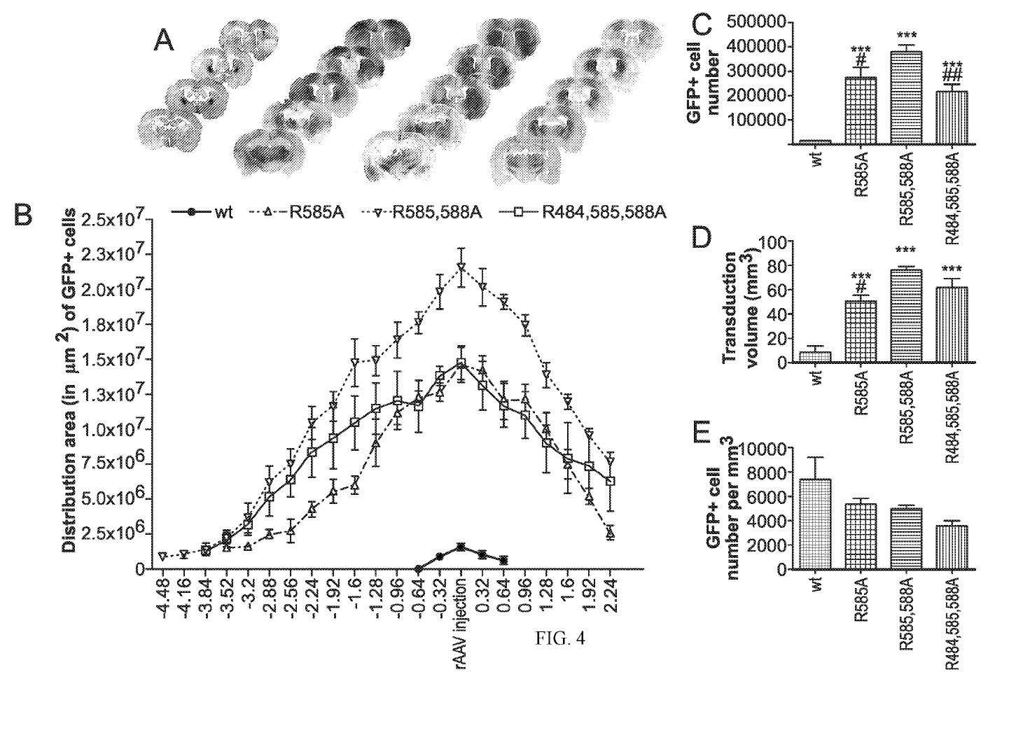

A non-limiting quantitative analysis of GFP-positive cells in the brain at 4 weeks after rAAV injections is depicted in FIG. 4. Montages of rostral-to-caudal coronal sections illustrate the extent of expression of GFP in the brain after bi-lateral injections with wt and capsid mutant viral vectors (FIG. 4A). Outlined areas (in μm²) containing GFP-positive cells on every 8 serial section were delineated for unbiased stereology count and demonstrate comparative distribution extent of cell infected with wt and capsid mutant viruses (FIG. 4B). As shown in FIG. 4C, stereological counts of transduced cells show that the number of GFP-expressing cells were vastly greater among the rAAV mutants than for the wild-type, with the double mutant having a significant increase over the other mutants. A similar trend was observable when assessing the volume of distribution through the brain tissue transduced with wt and capsid mutant viruses (FIG. 4D). The number of cells transduced per volume (mm³) calculated from measurements in FIGS. 4C and D is shown in FIG. 4E.

### Example 4: rAAV Variant Demonstrates Selective Transduction of Dopaminergic Neurons

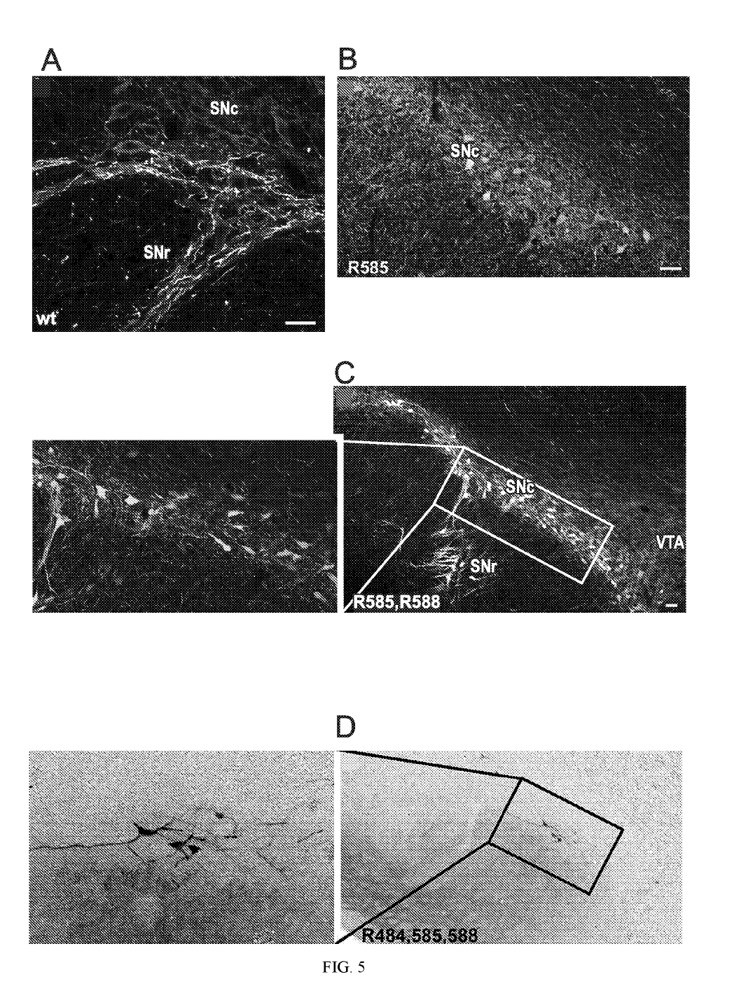

Antero-retrograde transport of wt and mutant rAAV was investigated to further assess transduction (FIG. 5). Merged views are depicted (FIG. 5A-C), showing TH-positive neurons that have been transduced with GFP. The wild-type rAAV showed GFP tracing of the anterograde projections to the substantia nigra pars reticulate (SNr), but no GFP expressing TH-positive cells in the substantia nigra pars compacta (SNc) were observed (FIG. 5A). Retrograde transport of the R585A mutant in the SNc was assessed (FIG. 5B). Confocal imaging demonstrated a selective transduction of dopaminergic neurons in the SNc after R585, 588A mutant injection into the striatum (FIG. 5C). Only single GFP expressing nigral neurons were detected with standard immune-peroxidase method in rats injected with R484, 585, 588A mutant virus (FIG. 5D). Notably, there were no TH-positive cells in the ventral tegmental area (VTA) expressing GFP or GFP-positive cells in any surrounding structure.

### Example 5: Subretinal Injection of Wild-Type and Variant rAAV

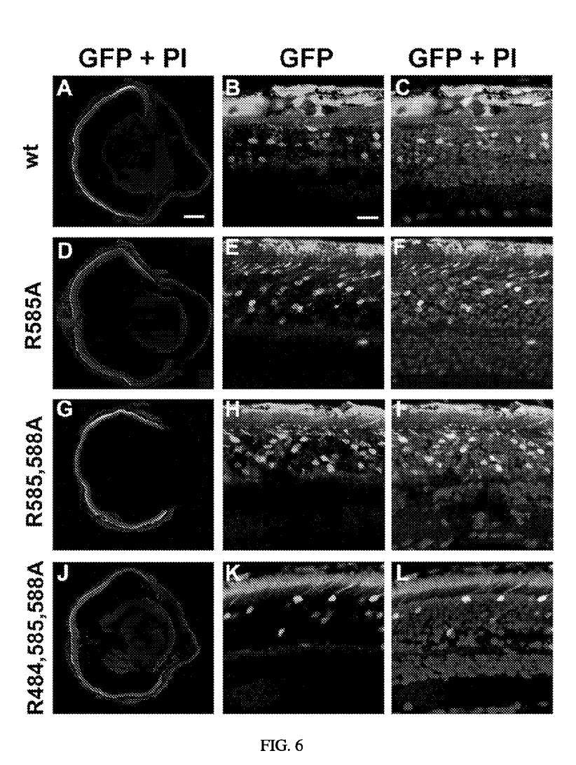

The results of wt, single, double, and triple variant distribution after subretinal injection are shown in FIG. 6. Full retinal mapping was conducted using propidium iodide (PI) nuclear staining and GFP native fluorescence (FIGS. 6A, D, G, and J). GFP-only fluorescence imaging (FIGS. 6B, E, H, and K) and GFP-PI overlaid imaging (FIGS. 6C, F, I, and L) were analyzed in representative retinal sectors. Confocal imaging demonstrated that virus expression was found in photoreceptors cells (PR) of injected retinas: the outer nuclear layer (ONL) and outer segments (OS). Additionally, all conditions demonstrated expression of GFP in retinal pigment epithelium (RPE). It was found that the transduction by wt, single, double, and triple mutants was not limited by the place of injection and extended across the full retina. However, it is necessary to mention that the density of viral transduction was not equal in all injected retinas.

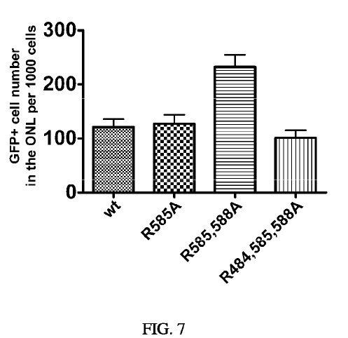

Results of the stereological counting of the GFP expressing PR cells in the representative segments of the ONL are shown in the FIG. 7. Stereological counts shown as a number of GFP-positive cells in ONL per 1000 propidium iodide labeled cells demonstrated that the number of transduced PR cells in retinas injected with wt, single mutant, and triple mutant was not significantly different (121±14.6, 127±16.6 and 101±14.1, respectively). The only exception found was the double mutant R585, 588A, which transduced a significantly higher number of PR cells. The counting of the GFP positive cells in the ONL of retinas injected with double mutant showed approximately two-fold difference compared to the others viruses (232±22.3; p<0.01 compared to wt and R585A and p<0.001 compared to R484, 585, 588A; N=4 for all animal groups).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method of delivering a gene of interest to a cell of the brain or eye, the method comprising providing to the cell a composition comprising a recombinant adeno-associated virus (rAAV) particle comprising an amino acid substitution at one or more positions selected from R484, R487, K532, R585, and R588.

2. The method of claim 1, wherein the recombinant adeno-associated virus (rAAV) particle comprises an amino acid substitution selected from R484A, R487A, K532A, R585A, and R588A.

3. The method of any of claims 1-2, wherein the rAAV particle is derived from an AAV2 serotype.

4. The method of any of claims 1-2, wherein the rAAV particle is derived from an AAV1 or AAV3 serotype.

5. The method of any of claims 1-4, wherein the rAAV particle comprises a nucleic acid encoding the gene of interest.

6. The method of claim 5, wherein the gene of interest is a therapeutic gene.

7. The method of claim 6, wherein the therapeutic gene encodes a therapeutic polypeptide or a therapeutic protein.

8. The method of claim 6, wherein the therapeutic gene encodes a therapeutic ribonucleic acid (RNA).

9. The method of claim 8, wherein the RNA comprises mRNA, tRNA, rRNA, siRNA, microRNA, antisense RNA, or a ribozyme.

10. The method of claim 6, wherein the therapeutic gene is a brain-specific gene or an eye-specific gene.

11. The method of claim 6, wherein the therapeutic gene comprises brain-derived neurotrophic factor (BDNF), tyrosine hydroxylase, aromatic amino acid decarboxylase, β-glucuronidase, exosaminidase A, herpes simplex virus, or thymidine kinase.

12. The method of claim 6, wherein the therapeutic gene comprises opsin protein of rhodopsin (RHO), cyclic GMP phosophodiesterase α-subunit (PDE6A) or β-subunit (PDE6B), alpha subunit of the rod cyclic nucleotide gated channel (CNGA1), RPE65, RLBP1, ABCR, peripherin/RDS, ROM1, arrestin (SAG), alpha-transducin (GNAT1), rhodopsin kinase (RHOK), guanylate cyclase activator 1A (GUCA1A), retina specific guanylate cyclase (GUCY2D), alpha subunit of the cone cyclic nucleotide gated cation channel (CNGA3), BCP cone opsin gene, GCP cone opsin gene, or RCP cone opsin gene.

13. An rAAV particle comprising:
a) an amino acid substitution at one or more positions selected from R484A, R487A, K532A, R585A, and R588A; and
b) a brain-specific or eye-specific gene of interest, or a gene of interest operatively connected to a brain-specific or eye-specific promoter.

14. A composition comprising the rAAV particle of claim 12.

15. The composition of claim 13, wherein the gene of interest is flanked by AAV inverted terminal repeats (ITRs).

16. A method of targeting the brain or eye of a subject, the method comprising administering to the subject a composition comprising a recombinant adeno-associated virus (rAAV) particle comprising an amino acid substitution at one or more positions selected from R484, R487, K532, R585, and R588.

17. The method of claim 16, wherein the rAAV particle comprises an amino acid substitution selected from R484A, R487A, K532A, R585A, and R588A

18. The method of claim 16, wherein the composition is administered subcutaneously, intraocularly, intravitreally, parenterally, subcutaneously, intravenously, intracranially, intracerebrally, intracerebro-ventricularly, intramuscularly, intrathecally, orally, intraperitoneally, intraspinally, epidurally, intradurally, subdurally, retrobulbarly, ophthalmicly, subretinally, intracorneally, conjunctivally, directly to the brain, directly to the CNS, directly to the peripheral nervous system, subconjunctivally, by oral or nasal inhalation, or by direct injection to one or more cells, tissues, or organs.

19. The method of claim 16, wherein the subject has a brain or eye condition, disease, or disorder.
